(19) United States
(12) Patent Application Publication
(10) Pub. No.: US 2016/0061698 A1
(43) Pub. Date: Mar. 3, 2016

(54) METHOD OF COLLECTING OF RARE CELLS FROM THE ENCLOSED FILTERS SYSTEM

(71) Applicant: HITACHI CHEMICAL COMPANY, LTD., Tokyo (JP)

(72) Inventors: Anthony H. TSAI, Irvine, CA (US); Kenji TAKAI, Tokyo (JP); Satomi YAGI, Tokyo (JP)

(21) Appl. No.: 14/838,885

(22) Filed: Aug. 28, 2015

Related U.S. Application Data
(60) Provisional application No. 62/043,446, filed on Aug. 29, 2014.

(51) Int. Cl. G01N 1/40; B01D 29/66; C12N 5/09

(52) U.S. Cl. CPC G01N 1/4077; C12N 5/0693; B01D 29/66; G01N 2001/4088; C12N 2509/00

(57) ABSTRACT

The methods of rare cells collection from enclosed micro filter system are provided. This invention includes surface treatment of the filter, application of cells dissociation reagent, shaking/vibration and back/side flush systems.

### BACKGROUND OF THE INVENTION

1. Field of the Invention

The present invention relates to cell collection method after rare cells captured on the micro filtration system.

2. Description of the Related Art

Circulating tumor cells (CTCs) are relatively rare cells which defined as tumor cells circulating in the peripheral blood of patients with metastatic cancer. Since metastases are directly related to the deaths of cancer patients, CTCs may compose the seeds for metastases and may be profitable for the spread of the cancers. Many studies showing that the appearance of CTCs in peripheral blood or bone marrow is a significant prognostic in different types of solid tumors. The number and molecular changes of CTCs may help to predict or monitor the response of treatment.

Current US Food and Drug Administration (FDA)-approved CellSearch system (Veridex, Raritan, N.J., USA) analysis is using magnetic beads coated with a monoclonal antibody-targeting epithelial cell marker, such as the epithelial cell-adhesion molecule (EpCAM) to enrich CTCs. This method does not capture all types of CTCs, and the recovery of the captured cells for the downstream molecular or cellular analysis is limited. Thus it is important to develop an improved methodology for CTC isolation that enables subsequent molecular analysis even at the single cell level in order to understand the origin and role of CTCs in cancer progression and treatment response.

Recently, devices using microfabrication method for size-based separation of tumor cells have been widely developed to enable precise and efficient enrichment of CTCs from whole blood. These devices include a miniaturized micro filter array system that can be used to trap rare cells by filtration based on differences in the sizes of cells. We have presented the capability of our micro filtration system by detecting the spiked tumor cells in human whole blood based on sizes and deformability between tumor cells and blood cells. It is important to collect the captured rare cells to enable CTC molecular analysis that will significantly contribute to cancer research and the selection of treatment options for patients based on changes in CTC numbers and molecular characteristics before and during treatment.

### SUMMARY OF THE INVENTION

The object of this invention is to effectively collect the cells in the enclosed micro filter system without breaking the enclosure. Surface treatment with albumin and derivatives before filtration is necessary to increase the cell recovery rate.

Following describes the process after filtration. First, we shake or vibrate the enclosed cartridge to release the cell from the filter surface. Cell disassociation reagent (eg. Trypsin or other serine proteases) may apply if the adhesion between cells and substrate is strong. Second, syringe with approximately 3 ml wash buffer is connected to the outlet of the cartridge, and syringe with approximately 1 ml wash buffer is connected to one inlet of the cartridge. Third, when injecting wash buffer from the above syringes the cells in the cartridge will be flushed out and collected in a clean container for further analysis.

Molecular analysis (eg. DNA, RNA . . . etc) could be applied afterward. The extended study could be single cell analysis after cell collection as well. The recovery rate of cell collection is higher than 70% and without damaging the cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other aspects of the invention will be readily apparent from the following description and from the appended drawings (not to scale), which are meant to illustrate and not to limit the invention, and wherein:

FIG. 1 illustrates the design of micro cavity array where 10a is the width (short pore) and 10b is the length (long pore) of the opening.

FIG. 2 is a cross-sectional view of cartridge.

FIG. 3A presents the cancer cells (arrow) on the substrate after filtration.

FIG. 3B shows the captured cancer cells move after shaking by comparing with FIG. 3A.

FIG. 4 explains cells collection from an enclosed cartridge which includes the buffer inlets and sample outlet.

FIG. 5 describes the process to confirm the cell collection. Cells collect by using the described method in FIG. 3, collect in a centrifuge tube, and then process through MCA system.

FIG. 6A is the image of cells on the micro cavity array before cell collection.

FIG. 6B is the image of cells on a new micro cavity array after processing the collected cells solution.

FIG. 6C is the image of cells on original micro cavity array after cell collection process.

FIG. 7 demonstrates direct cell collection design through MCA system without centrifuge tube transferring.

FIG. 8A is the image of cells on the micro cavity array before cell collection.

FIG. 8B is the image of cells on the new micro cavity array by using direct collection then processing the collected cells solution.

FIG. 9 is the table showing the recovery rate by using cell collection method through centrifuge collection and direct collection.

### DETAILED DESCRIPTION OF THE EMBODIMENTS

The following detailed description is directed to certain specific embodiments of the invention. However, the invention can be embodied in a multitude of different ways. In this description, reference is made to the drawing wherein like parts are designated with like numerals throughout.

Embodiments of methods of present invention are designed to effectively recover the captured rare cells from the enclosed filter substrate. The process is simple, less expensive and more efficient.

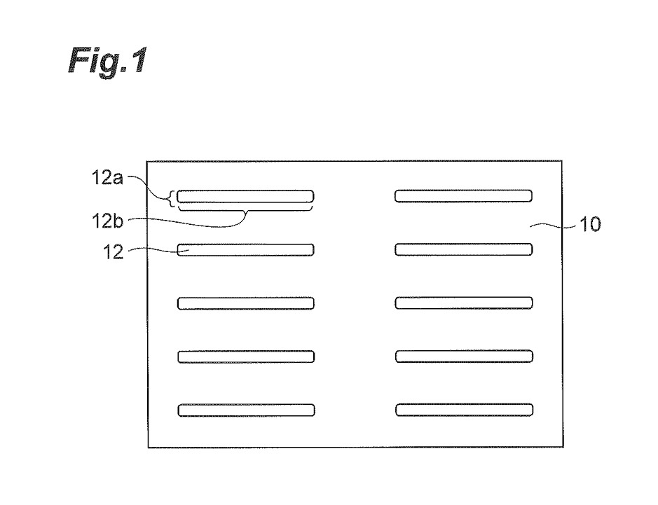

FIG. 1 depicts certain embodiments of the filter comprising a substrate 10 and at least one aperture 12. Generally substrate 10 may be any material that can form a rigid film including but not limited to metal and non-metal materials. Non-limiting examples of substrate materials are metal material such as Au, Pt, Ni, Cu, Pd and non-metal material such as carbon, polymer, ceramic, semiconductor, and composite material. The size of the aperture 12 determined by the width 12a and the length 12b where 12a is about 1 μm to about 20 μm, preferably about 5 μm to about 12 μm, more preferably about 7-10 μm and 12b is about 5 μm to about 1000 μm, preferably about 10 μm to about 500 μm, more preferably about 50-200 μm.

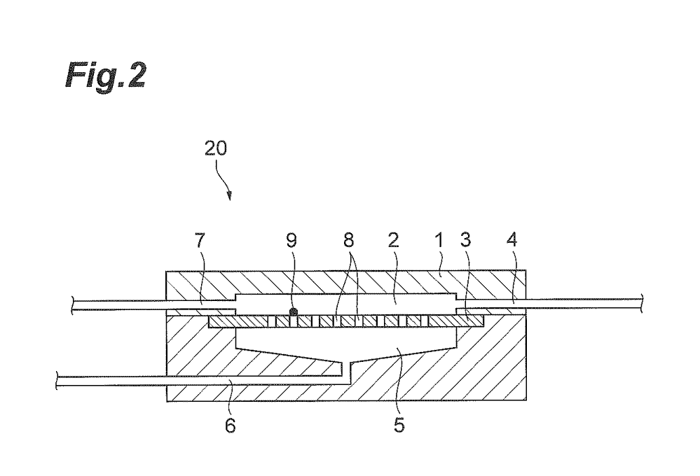

FIG. 2 is a cross-sectional view of cartridge 20. As shown in FIG. 2, a cartridge 20 includes a housing 1 that includes a first inlet 4 and a second inlet 7 through which a cell dispersion or the other liquid is introduced and an outlet 6 through which the cell dispersion liquid or other liquid is discharged. The Filter 3 which is positioned within the housing 1 includes at least one aperture 8 (through-hole). In the housing 1, upper space 2 is formed on the filter 3 and lower space 5 is formed under the filter.

When a cell is captured on the filter 3, generally, blood (of the cell dispersion liquid) is introduced through a first inlet 4 of the cartridge 20, passed the filter 3, and discharged through the outlet 6. Next, a reagent (e.g. stain solution) is introduced through a second inlet 7 of the cartridge 20, passed the filter 3, and discharged through the outlet 6. The trapping (filtration) and treatment of cells 9 are carried out by the above method.

There are two method to collect cells 9 from the filter 3. The first is to move a wash buffer from the outlet 6 to the second inlet 7 and to collect by washing cells 9 away. This first method is called as “back flash”. The second is to move a wash buffer from the first inlet 4 to the second inlet 7 to collect by washing cells 9 away. This second method is called as “side flash”

Some rare cells, such as breast cancer (MCF7, SKBR3), lung cancer (NCI-H358, A549), liver cancer (Hep G2), colon cancer (HT29) and melanoma (A375), tend to adhere to the substrate after filtration, certain pretreatment to the surface of the substrate and post treatments after filtration are necessary in order to collect the captured cells.

Surface pretreatment materials including genuine and artificial materials have to biocompatible for this application. Typically genuine materials for the application include but not limited to protein and derivatives, preferably albumin which includes but not limited to bovine, mouse chicken egg and human. Examples of artificial biocompatible materials are Poly(methyl methacrylate) (PMEA) and 2-methacryloyloxyethyl phosphorylcholine (MPC).

Post treatment after filtration includes physically, chemically or combination methods.

Physical treatment includes but not limited to shake or vibrate the filter enclosure to remove the cells from the substrate, and flush out the cells from the back/side of the filter enclosure.

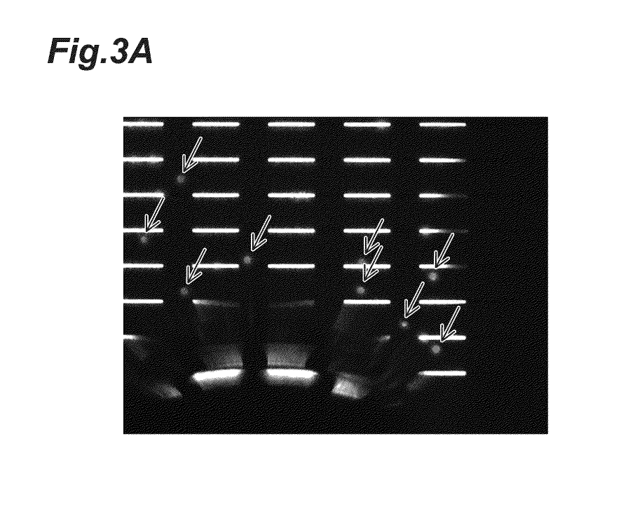

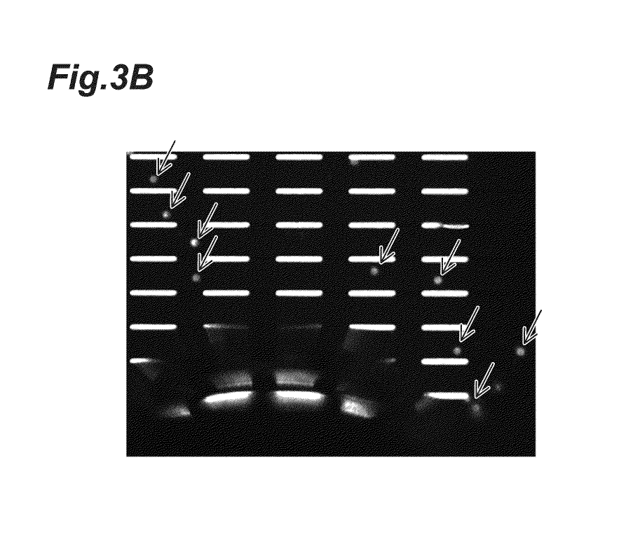

FIG. 3A shows cytokeratin stained lung cancer cells specifically NCI-11358 (red arrows) on the filter after filtration under fluorescence microscope. While FIG. 3B demonstrates the captured cells (red arrows) move after shaking the cartridge which means the cells detach from the substrate after shaking.

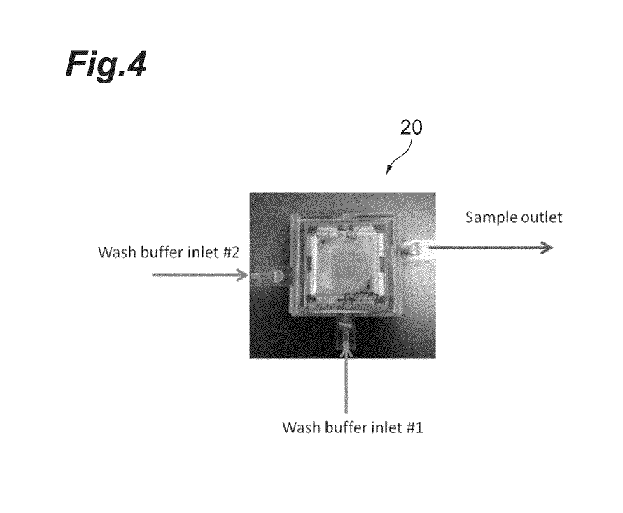

Another important process is back/side flush through the filter enclosure showing in FIG. 4 where wash buffer inlet #1 (an outlet 6 of FIG. 2) is from the back side of the filter, wash buffer inlet #2 (a first inlet 4 of FIG. 2) is from the top side of the filter as well as the sample outlet (a second inlet 7 of FIG. 2).

Wash buffer can be any biocompatible fluid not causing cell damages. An example of wash buffer is phosphate buffered saline (PBS) including Potassium Phosphate monobasic (KH₂PO₄) 1.06 mM, Sodium Chloride (NaCl) 155.17 mM, Sodium Phosphate dibasic (Na₂HPO₄—7H₂O) 2.97 mM in deionized water (D.I. water) along with bovine serum albumin (BSA) 0.5 mM and ethylenediaminetetraacetic acid (EDTA) 2 mM. The flow rate from wash buffer inlet can be controlled but not limited by manual with timer or micro syringe pump precisely.

The flow rate from buffer inlet #1 is about 0.2 ml/min to about 10 ml/min, preferably about 0.5 ml/min to about 5 ml/min, more preferably about 1 ml/min to about 3 ml/min. While flow rate from buffer inlet #2 is about 0.1 ml/min to about 5 ml/min, preferably about 0.2 ml/min to about 3 ml/min, more preferably 0.3 ml/min to 1 ml/min.

Volume of wash buffer from inlet # 1 is about 0.2 ml to about 20 ml, preferably about 0.5 ml to about 10 ml, more preferably about 1 ml to 5 ml. Volume of wash buffer from inlet #2 is about 0.1 ml to about 10 ml, preferably about 0.2 ml to about 5 ml, more preferably about 0.5 ml to about 3 ml.

In some cases the adhesion between cells and substrate is very strong, cell disassociation reagent including but not limited to serine proteases (eg. trypsin) along with incubator can be applied before back/side flush process.

If necessary, cell disassociation reagent can be injected from inlet #1 and the exceeded solution will be collected from outlet then add to the later back/side flush solution.

The volume of cell disassociation reagent is about 100 μl to about 5 ml, preferably about 200 μl to about 3 ml, more preferably about 500 μl to about 2 ml.

The cell disassociation reagent injected cartridge is then put into the incubator.

The temperature setup of the incubator is about 25° C. to about 50° C., preferably about 30° C. to about 45° C., more preferably about 35° C. to about 38° C.

The incubation time is about 0 minute to about 10 minutes, preferably about 1 minute to about 7 minutes, more preferably about 2 minutes to 5 minutes.

### EXAMPLE 1

Cell collection in centrifuge tube: Surface of the filter was pretreated with priming buffer (potassium phosphate monobasic (KH₂PO₄) 1.06 mM, sodium chloride (NaCl) 155.17 mM, sodium phosphate dibasic (Na₂HPO₄—7H₂O) 2.97 mM in deionized water (D.I. water) along with fetal bovine serum (FBS) 20% and ethylenediaminetetraacetic acid (EDTA) 2 mM) for at least 5 minutes.

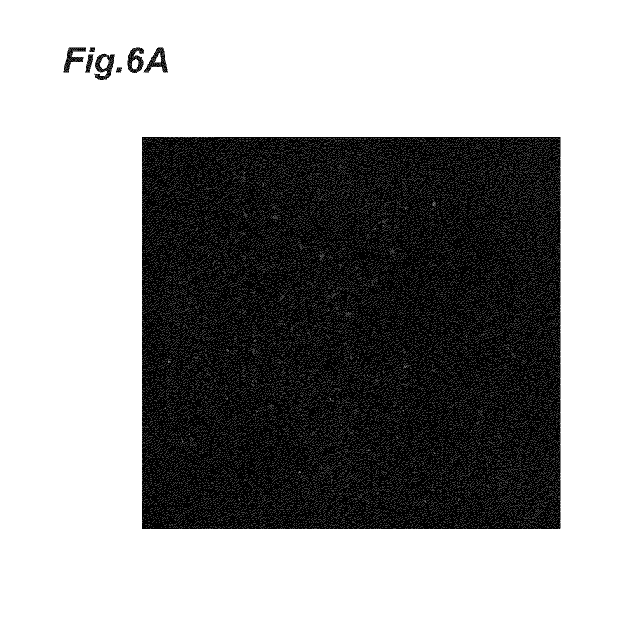

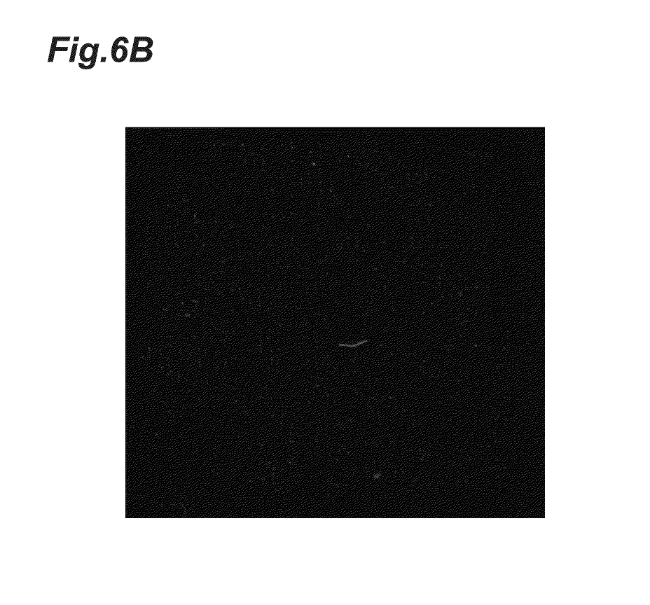

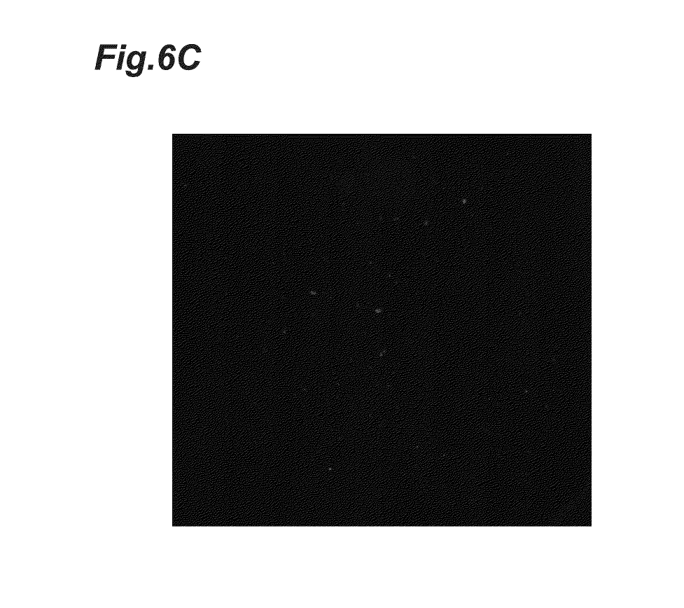

About 1000 NCI-H358 cancer cells in the same wash buffer was spike into 3 ml peripheral blood then process through CT6000 (Hitachi Chemical CTC capturing system) which includes fixing/lysing, permeabilization, cell staining and washing processes. The captured and stained cells on the filter are showing in FIG. 6A.

The enclosed filter unit was then shaken and processed the back/side flush procedure described in FIG. 4.

An about 3 ml wash buffer (Potassium Phosphate monobasic (KH₂PO₄) 1.06 mM, Sodium Chloride (NaCl) 155.17 mM, Sodium Phosphate dibasic (Na₂HPO₄—7H₂O) 2.97 mM in deionized water (D.I. water) along with bovine serum albumin (BSA) 0.5 mM and ethylenediaminetetraacetic acid (EDTA) 2 mM) was injected from wash buffer inlet #1 with about 1.5 ml/min flow rate and same wash buffer was injected from wash buffer inlet #2 with about 0.5 ml/min flow rate at the same time.

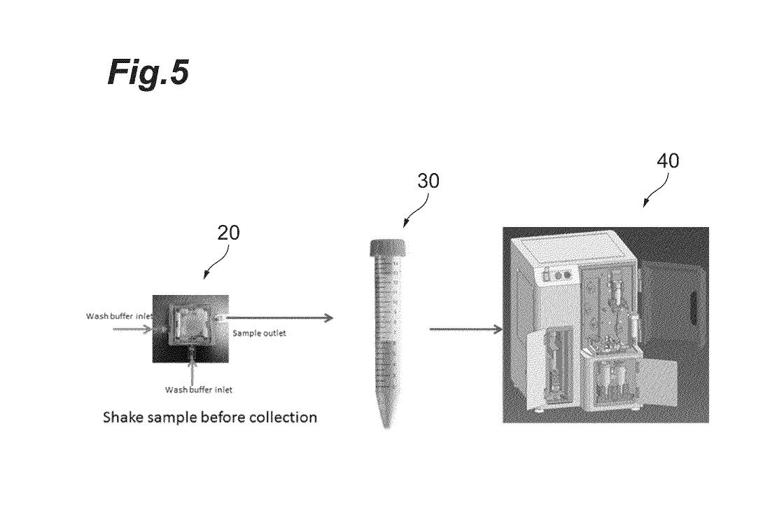

The solution (about 4 ml) from sample outlet was collected in a 15 ml centrifuge tube 30 then transferred into a syringe in CT6000 (MCA system 40) as shown in FIG. 5.

The collected solution was processed through the CT6000 system with new filter cartridge by using only filtration process (no fixing, permeabilization and staining).

FIG. 5B shows the captured cells after back/side flush cell collection process with about 71% recovery rate compared with the original captured cells in FIG. 6A.

The original filter unit was also examined after back/side flush process showing in FIG. 6C.

The remaining cells on original filter are only about 3% which means 97% of the cells were successfully removed from the filter unit.

Rest of the cells might be lost during transferring from cartridge to centrifuge tube, from centrifuge tube to the syringe in CT6000 or/and during second filtration process.

### EXAMPLE 2

Direct cell collection: Surface of the filter was pretreated with priming buffer (potassium phosphate monobasic (KH₂PO₄) 1.06 mM, sodium chloride (NaCl) 155.17 mM, sodium phosphate dibasic (Na₂HPO₄—7H₂O) 2.97 mM in deionized water (D.I. water) along with fetal bovine serum (FBS) 20% and ethylenediaminetetraacetic acid (EDTA) 2 mM) for at least 5 minutes.

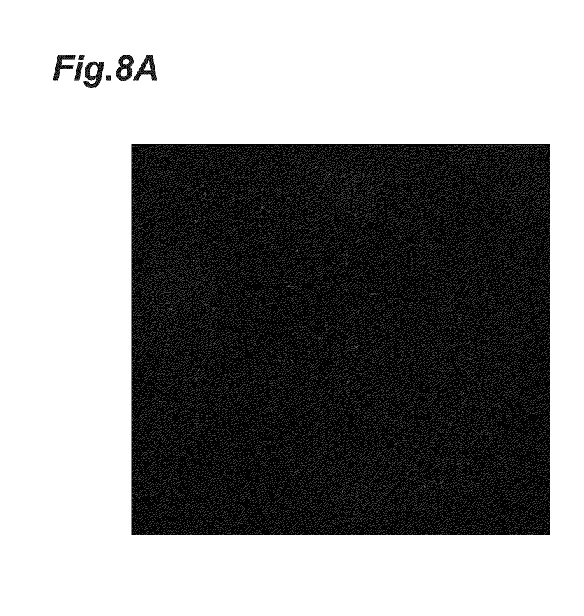

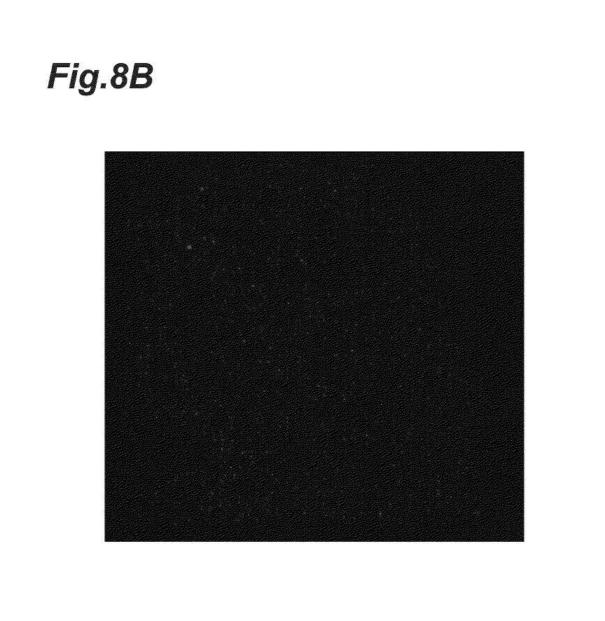

About 1000 NCI-H358 cancer cells in the same wash buffer was spike into 3 ml peripheral blood then process through CT6000 which includes fixing/lysing, permeabilization, cell staining and washing processes. The captured and stained cells on the filter are showing in FIG. 8A.

The enclosed filter unit was then shaken and processed the back/side flush procedure described in FIG. 4.

An about 3 ml wash buffer (Potassium Phosphate monobasic (KH₂PO₄) 1.06 mM, Sodium Chloride (NaCl) 155.17 mM, Sodium Phosphate dibasic (Na₂HPO₄—7H₂O) 2.97 mM in deionized water (D.I. water) along with bovine serum albumin (BSA) 0.5 mM and ethylenediaminetetraacetic acid (EDTA) 2 mM) was injected from wash buffer inlet #1 with about 1.5 ml/min flow rate and same wash buffer was injected from wash buffer inlet #2 with about 0.5 ml/min flow rate at the same time.

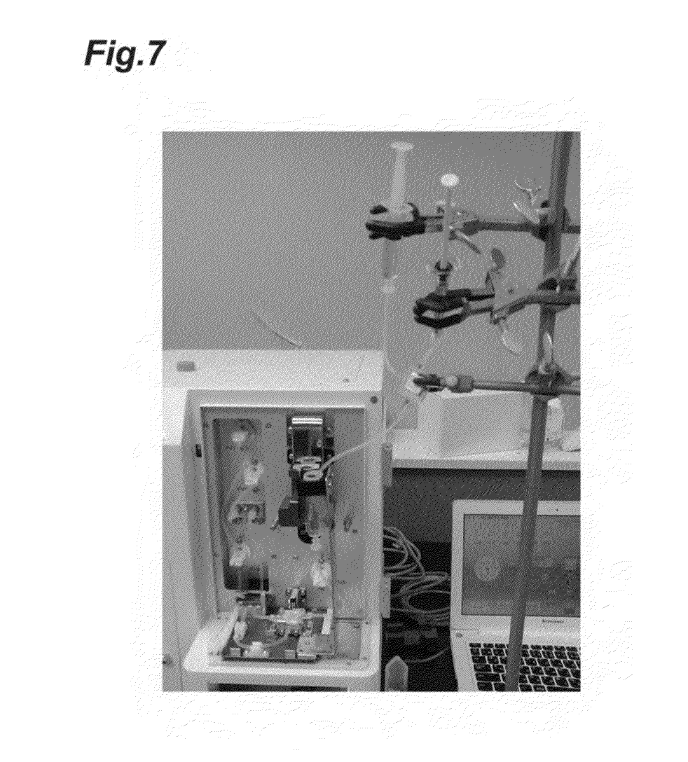

The solution (about 4 ml) from sample outlet was directly collected to the syringe in CT6000 system as shown in FIG. 7.

The collected solution was processed through the CT6000 system with new filter cartridge by using only filtration process (no fixing, permeabilization and staining).

FIG. 8B shows the captured cells after back/side flush cell collection process with about 76% recovery rate compared with the original captured cells in FIG. 7A.

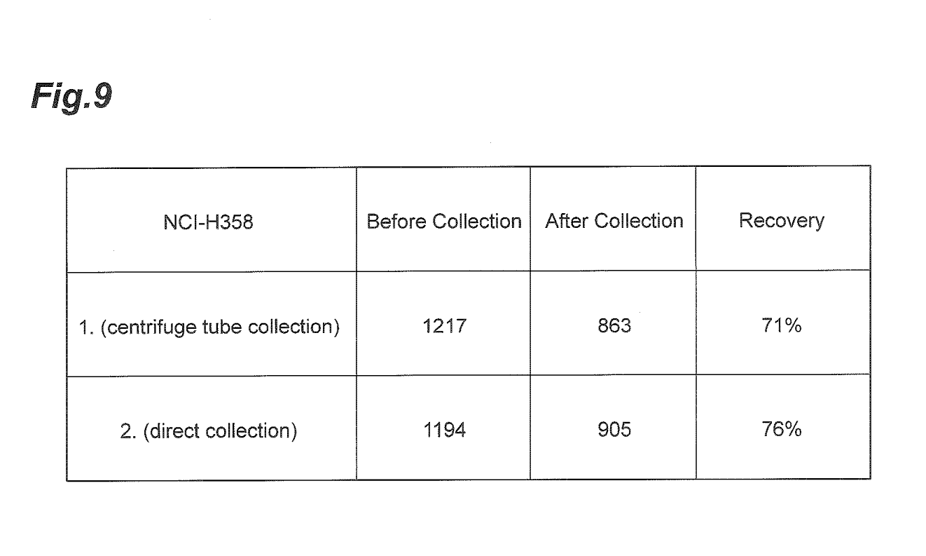

FIG. 9 is the table showing the cell recovery by using two different collection methods. Direct collection method cell recovery rate increase about 5% compared with centrifuge transferring.

What is claimed is:

1. A method of collecting captured rare cells from an enclosed filter system, comprising;
surface pretreatment, shaking and/or vibration, and back/side flush to collect captured rare cells from the enclosed filter system.

2. The method of collecting captured rare cells from the enclosed filter system according to claim 1 wherein the surface pretreatment materials include genuine and artificial materials.

3. The method of collecting captured rare cells from the enclosed filter system according to claim 1 wherein the genuine materials using in the surface pretreatment is protein and derivatives, especially albumin which includes bovine, mouse chicken egg and human.

4. The method of collecting captured rare cells from the enclosed filter system according to claim 1 wherein the artificial materials using in surface pretreatment are Poly(methyl methacrylate) (PMEA) and 2-methacryloyloxyethyl phosphorylcholine (MPC).

5. The method of collecting captured rare cells from the enclosed filter system according to claim 1 wherein the treatment time of the surface pretreatment is at least 1 minute.

6. The method of collecting captured rare cells from the enclosed filter system according to claim 1 wherein the shaking method can be manual or shaker.

7. The method of collecting captured rare cells from the enclosed filter system according to claim 1 wherein the vibration method can be manual or shaker.

8. The method of collecting captured rare cells from the enclosed filter system according to claim 1 wherein back/side flush method comprising of flushing captured cells from two wash buffer inlets.

9. The method of collecting captured rare cells from the enclosed filter system from according to claim 8 wherein the volume of wash buffer from inlet #1 is 1 ml to 5 ml.

10. The method of collecting captured rare cells from the enclosed filter system according to claim 8 wherein the flow rate of wash buffer from inlet #1 is 1 ml/min to 5 ml/min.

11. The method of collecting captured rare cells from the enclosed filter system according to claim 8 wherein the volume of wash buffer from inlet #2 is 0.5 ml to 3 ml.

12. The method of collecting captured rare cells from the enclosed filter system according to claim 8 wherein the flow rate of wash buffer from inlet #2 is 0.5 ml/min to 3 ml/min.

13. The method of collecting captured rare cells from the enclosed filter system according to claim 1 wherein the method including cell dissociation reagent injection and incubation.

14. The method of collecting captured rare cells from the enclosed filter system according to claim 13 wherein the cell dissociation reagent is serine proteases.

15. The method of collecting captured rare cells from the enclosed filter system according to claim 13 wherein the injection volume of cell dissociation reagent should be 500 μl to 2 ml.

16. The method of collecting captured rare cells from the enclosed filter system according to claim 13 wherein the incubation temperature of injected cell dissociation reagent should be 35° C. to 38° C.

17. The method of collecting captured rare cells from the enclosed filter system according to claim 13 wherein the incubation time of injected cell dissociation reagent should be 2-5 minutes.

18. The method of collecting captured rare cells from the enclosed filter system according to claim 1 wherein some process are carried out in continuity.
